(19) United States
(12) Patent Application Publication
(10) Pub. No.: US 2026/0174548 A1
(43) Pub. Date: Jun. 25, 2026

(54) ACCOMMODATING INTRAOCULAR LENSES WITH PHASE SHIFTING LENS SURFACE PROFILE, INTRAOCULAR LENSES WITH MECHANICAL ACCOMMODATING STRUCTURE, AND METHODS OF USE THEREOF

(71) Applicant: Alcon Inc., Fribourg (CH)

(72) Inventors: Iulen CABEZA-GIL, Foster City, CA (US); Robert Dimitri ANGELOPOULOS, San Jose, CA (US); Terah Whiting SMILEY, Davis, CA (US); Nathan LEWIS, San Jose, CA (US)

(73) Assignee: Alcon Inc., Fribourg (CH)

(21) Appl. No.: 19/408,065

(22) Filed: Dec. 3, 2025

Related U.S. Application Data
(60) Provisional application No. 63/737,445, filed on Dec. 20, 2024.

(51) Int. Cl. A61F 2/16

(52) U.S. Cl. CPC A61F 2/1635; A61F 2/1648; A61F 2002/1682; A61F 2002/16901

(57) ABSTRACT

Disclosed are accommodating intraocular lenses with a phase shifting lens surface profile, intraocular lenses with a mechanical accommodating structure, and methods of use thereof. For example, an intraocular lens is disclosed comprising an optic portion comprising an anterior lens and a posterior lens and an accommodating mechanical structure coupled to the optic portion. The anterior lens can comprise a phase shifting lens surface profile. The mechanical accommodating structure can comprise a plurality of compressible haptics. Each of the compressible haptics can be shaped as an annulus sector and can comprise a fluted or plicated portion configured to allow the haptics to compress or expand.

### CROSS-REFERENCE TO RELATED APPLICATION

This application claims the benefit of and priority to U.S. Provisional Ser. No. 63/737,445 filed on Dec. 20, 2024, the content of which is incorporated herein by reference in its entirety.

### TECHNICAL FIELD

The present disclosure relates generally to the field of intraocular lenses, and, more specifically, to accommodating intraocular lenses with a phase shifting lens surface profile, intraocular lenses with a mechanical accommodating structure, and methods of use thereof.

### BACKGROUND

Presbyopia is an age-related condition affecting the eyes' ability to focus on nearby objects. By the age of 50, nearly everyone experiences some degree of presbyopia. It is estimated that around 1.8 billion people worldwide suffers from presbyopia and this number is expected to grow as the global population ages. Presbyopia does not indicate any underlying health issues but the condition can be frustrating for those who require clear near-vision for work or daily activities. People generally address presbyopia by relying on reading glasses.

However, even after undergoing cataract surgery, presbyopia still persists as a problem, significantly impacting people's quality of life. One way to address this problem is during cataract surgery by implanting an intraocular lens that can restore a wide range of vision (e.g., near, intermediate, and distance) without the need for reading glasses. However, traditional intraocular lenses often are not designed to replicate the eye's natural accommodating mechanism and may even introduce visual disturbances like halos or glares.

Therefore, a solution is needed that can replicate the eye's natural accommodating mechanism and minimize unwanted visual disturbances such as halos or glares. Such a solution should not be overly complicated and still allow the IOL to be cost-effectively manufactured.

### SUMMARY

Disclosed herein are accommodating intraocular lenses with a phase shifting lens surface profile, intraocular lenses with a mechanical accommodating structure, and methods of use thereof. In some embodiments, an intraocular lens is disclosed comprising an optic portion and an accommodating mechanical structure coupled to the optic portion. The optic portion can comprise an anterior lens and a posterior lens.

In some embodiments, the optic portion can comprise only the anterior lens or the posterior lens can be a non-optical component or non-optical lens.

At least one of the anterior lens and the posterior lens can comprise an optical surface profile. For example, the anterior lens can comprise the optical surface profile. Also, for example, the posterior lens can comprise the optical surface profile. In some embodiments, the optical surface profile can be a phase-shifting surface profile or a presbyopia-correcting surface profile.

In some embodiments, the anterior lens is unconnected to the posterior lens other than via the accommodating mechanical structure.

In some embodiments, the intraocular lens has an accommodated configuration and a non-accommodated configuration. The intraocular lens can be axially compressed when in the non-accommodated configuration and axially expanded when in the accommodated configuration.

In some embodiments, an axial distance separating the anterior lens and the posterior lens can be increased when the intraocular lens is in the accommodated configuration compared to when the intraocular lens is in the non-accommodated configuration.

In some embodiments, the axial distance separating the anterior lens and the posterior lens can be increased by about 500 μm when the intraocular lens is in the accommodated configuration compared to when the intraocular lens is in the non-accommodated configuration. The accommodating mechanical structure and the optic portion are unfilled with a fluid.

In some embodiments, the accommodating mechanical structure can comprise a plurality of compressible haptics. For example, the compressible haptics can comprise a first compressible haptic and a second compressible haptic. The first compressible haptic can be positioned diametrically opposed to the second compressible haptic.

In some embodiments, each of the compressible haptics can be substantially shaped as an annulus sector or partial annulus when viewing the intraocular lens in an anteroposterior direction. In these embodiments, none of the compressible haptics is shaped as a semi-annulus when viewing the intraocular lens in an anteroposterior direction (or top down direction when the intraocular lens is not implanted).

In some embodiments, each of the compressible haptics can comprise an anterior haptic portion, a posterior haptic portion, and a connecting portion configured to couple the anterior haptic portion to the posterior haptic portion.

In some embodiments, the connecting portion can comprise cutouts or openings along the connecting portion.

In some embodiments, the connecting portion can be fluted or plicated in an anteroposterior direction. The fluted or plicated surface of the connecting portion can face radially outward from the optic portion.

In some embodiments, the connecting portion can comprise a first curved flute portion and a second curved flute portion connected by an elongate fold. At least one of the first curved flute portion and the second curved flute portion can comprise three cutouts or openings.

In some embodiments, the anterior lens can be convergent (a positive power lens). In these and other embodiments, the posterior lens can be divergent (a negative power lens).

In some embodiments, the intraocular lens can be made at least in part of a hydrophobic acrylic material.

Also disclosed is an accommodating intraocular lens comprising an optic portion and a plurality of haptics coupled to the optic portion. The optic portion can comprise an anterior lens and a posterior lens and the haptics can be configured to be compressible and expandable in an anteroposterior direction.

In some embodiments, the optic portion can comprise only the anterior lens or the posterior lens can be a non-optical component or non-optical lens.

In some embodiments, the anterior lens is unconnected to the posterior lens other than via the haptics. Moreover, the accommodating intraocular lens (including the optic portion) is unfilled with a fluid.

In some embodiments, the accommodating intraocular lens has an accommodated configuration and a non-accommodated configuration. The accommodating intraocular lens can be axially compressed when in the non-accommodated configuration. The accommodating intraocular lens can be axially expanded when in the accommodated configuration.

In some embodiments, an axial distance separating the anterior lens and the posterior lens can be increased when the accommodating intraocular lens is in the accommodated configuration compared to when the accommodating intraocular lens is in the non-accommodated configuration. For example, the axial distance separating the anterior lens and the posterior lens can be increased by about 500 μm when the accommodating intraocular lens is in the accommodated configuration compared to when the accommodating intraocular lens is in the non-accommodated configuration.

In some embodiments, the accommodating intraocular lens can comprise a first compressible haptic and a second compressible haptic. The first compressible haptic can be positioned diametrically opposed to the second compressible haptic.

In some embodiments, each of the haptics can be substantially shaped as an annulus sector or partial annulus when viewing the accommodating intraocular lens in an anteroposterior (or top down) direction. In these embodiments, none of the haptics is shaped as a semi-annulus when viewing the accommodating intraocular lens in an anteroposterior (or top down) direction.

In some embodiments, each of the haptics comprises an anterior haptic portion, a posterior haptic portion, and a connecting portion configured to couple the anterior haptic portion to the posterior haptic portion. The connecting portion can comprise cutouts or openings along the connecting portion.

In some embodiments, the connecting portion can be fluted or plicated in an anteroposterior direction. A fluted or plicated surface of the connecting portion can face radially outward from the optic portion.

In some embodiments, the connecting portion can comprise a first curved flute portion and a second curved flute portion connected by an elongate fold. At least one of the first curved flute portion and the second curved flute portion can comprise three cutouts or openings.

In some embodiments, the anterior lens can be convergent (a positive power lens). In these and other embodiments, the posterior lens can be divergent (a negative power lens).

In some embodiments, the accommodating intraocular lens can be made at least in part of a hydrophobic acrylic material.

In some embodiments, the anterior lens can comprise an optical surface profile. For example, the optical surface profile can be a phase-shifting surface profile or a presbyopia-correcting surface profile.

In some embodiments, the posterior lens can comprise the optical surface profile. For example, the posterior lens can comprise a phase-shifting surface profile or a presbyopia-correcting surface profile.

Also disclosed is an accommodating intraocular lens comprising an optic portion and at least one haptic coupled to the optic portion substantially shaped as an annulus sector. The at least one haptic can be substantially shaped as the annulus sector when viewing the accommodating intraocular lens in an anteroposterior (or top down) direction.

Moreover, none of the haptics is shaped as a semi-annulus when viewing the accommodating intraocular lens in an anteroposterior (or top down) direction.

The optic portion can comprise an anterior lens and a posterior lens. The anterior lens can be unconnected to the posterior lens other than via the haptics.

In some embodiments, the optic portion can comprise only the anterior lens or the posterior lens can be a non-optical component or non-optical lens.

In some embodiments, the accommodating intraocular lens has an accommodated configuration and a non-accommodated configuration. The accommodating intraocular lens can be axially compressed when in the non-accommodated configuration. The accommodating intraocular lens can be axially expanded when in the accommodated configuration.

In some embodiments, an axial distance separating the anterior lens and the posterior lens can be increased when the accommodating intraocular lens is in the accommodated configuration compared to when the accommodating intraocular lens is in the non-accommodated configuration. For example, the axial distance separating the anterior lens and the posterior lens can be increased by about 500 μm when the accommodating intraocular lens is in the accommodated configuration compared to when the accommodating intraocular lens is in the non-accommodated configuration. The accommodating intraocular lens (including the optic portion) is unfilled with a fluid.

In some embodiments, the accommodating intraocular lens can comprise a first haptic and a second haptic. The first haptic can be positioned diametrically opposed to the second haptic.

In some embodiments, each of the haptics can comprise an anterior haptic portion, a posterior haptic portion, and a connecting portion configured to couple the anterior haptic portion to the posterior haptic portion. The connecting portion can comprise cutouts or openings along the connecting portion.

In some embodiments, the connecting portion can be fluted or plicated in an anteroposterior direction. A fluted or plicated surface of the connecting portion can face radially outward from the optic portion.

In some embodiments, the connecting portion can comprise a first curved flute portion and a second curved flute portion connected by an elongate fold. At least one of the first curved flute portion and the second curved flute portion can comprise three cutouts or openings.

In some embodiments, the anterior lens can be convergent (a positive power lens). In these and other embodiments, the posterior lens can be divergent (a negative power lens).

In some embodiments, the accommodating intraocular lens can be made at least in part of a hydrophobic acrylic material.

At least one of the anterior lens and the posterior lens can comprise an optical surface profile. For example, the anterior lens can comprise the optical surface profile. Also, for example, the posterior lens can comprise the optical surface profile. In some embodiments, the optical surface profile can be a phase-shifting surface profile or a presbyopia-correcting surface profile.

Also disclosed is an accommodating intraocular lens comprising an optic portion and a plurality of haptics coupled to the optic portion. The optic portion can comprise an anterior lens and a posterior lens. At least one of the haptics can comprise an anterior haptic portion and a posterior haptic portion. The anterior haptic portion can be coupled to the posterior haptic portion by a connecting portion. The connecting portion can be fluted or plicated in an anteroposterior direction.

In some embodiments, the anterior lens is unconnected to the posterior lens other than via the haptics.

In some embodiments, the accommodating intraocular lens can have an accommodated configuration and a non-accommodated configuration. The accommodating intraocular lens can be axially compressed when in the non-accommodated configuration and axially expanded when in the accommodated configuration.

In some embodiments, an axial distance separating the anterior lens and the posterior lens can be increased when the accommodating intraocular lens is in the accommodated configuration compared to when the accommodating intraocular lens is in the non-accommodated configuration. For example, the axial distance separating the anterior lens and the posterior lens can be increased by about 500μm when the accommodating intraocular lens is in the accommodated configuration compared to when the accommodating intraocular lens is in the non-accommodated configuration. The accommodating intraocular lens (including the optic portion) is unfilled with a fluid.

In some embodiments, the accommodating intraocular lens can comprise a first haptic and a second haptic. The first haptic can be positioned diametrically opposed to the second haptic.

In some embodiments, each of the haptics can be substantially shaped as an annulus sector when viewing the accommodating intraocular lens in an anteroposterior direction. In these embodiments, none of the haptics is shaped as a semi-annulus when viewing the accommodating intraocular lens in an anteroposterior direction.

In some embodiments, the connecting portion can comprise cutouts or openings along the connecting portion. A fluted or plicated surface of the connecting portion can face radially outward from the optic portion.

In some embodiments, the connecting portion can comprise a first curved flute portion and a second curved flute portion connected by an elongate fold. At least one of the first curved flute portion and the second curved flute portion can comprise three cutouts or openings.

In some embodiments, the anterior lens can be convergent (a positive power lens). In these and other embodiments, the posterior lens can be divergent (a negative power lens). In some embodiments, the anterior lens can be convex and the posterior lens can be concave.

In some embodiments, the accommodating intraocular lens can be made at least in part of a hydrophobic acrylic material.

At least one of the anterior lens and the posterior lens can comprise an optical surface profile. For example, the anterior lens can comprise the optical surface profile. Also, for example, the posterior lens can comprise the optical surface profile. In some embodiments, the optical surface profile can be a phase-shifting surface profile or a presbyopia-correcting surface profile.

Also disclosed is an intraocular lens comprising an optic portion and an accommodating mechanical structure coupled to the optic portion. The optic portion can comprise an anterior lens and a posterior lens. The anterior lens can be a convergent lens and the posterior lens can be a divergent lens.

In some embodiments, the optic portion can comprise only the anterior lens or the posterior lens can be a non-optical component or non-optical lens.

In some embodiments, the anterior lens can be unconnected to the posterior lens other than via the accommodating mechanical structure.

In some embodiments, the intraocular lens can have an accommodated configuration and a non-accommodated configuration. The intraocular lens can be axially compressed when in the non-accommodated configuration and axially expanded when in the accommodated configuration.

In some embodiments, an axial distance separating the anterior lens and the posterior lens can be increased when the intraocular lens is in the accommodated configuration compared to when the intraocular lens is in the non-accommodated configuration. For example, the axial distance separating the anterior lens and the posterior lens can be increased by about 500 μm when the intraocular lens is in the accommodated configuration compared to when the intraocular lens is in the non-accommodated configuration.

In some embodiments, the accommodating mechanical structure can comprise a plurality of compressible haptics. Each of the compressible haptics can be substantially shaped as an annulus sector when viewing the intraocular lens in an anteroposterior (or top down) direction. The accommodating mechanical structure and the optic portion are unfilled with a fluid.

In some embodiments, the anterior lens can be convex and the posterior lens can be concave.

At least one of the anterior lens and the posterior lens can comprise an optical surface profile. For example, the anterior lens can comprise the optical surface profile. Also, for example, the posterior lens can comprise the optical surface profile.

Also disclosed is a method of correcting presbyopia in a subject. The method can comprise implanting an accommodating intraocular lens in the subject. The accommodating intraocular lens can comprise an optic portion and a mechanical structure coupled to the optic portion and configured to be compressible and expandable. The optic portion can comprise an anterior lens and a posterior lens.

In some embodiments, the anterior lens is unconnected to the posterior lens other than via the mechanical structure.

In some embodiments, the accommodating intraocular lens has an accommodated configuration and a non-accommodated configuration. The accommodating intraocular lens can be axially compressed when in the non-accommodated configuration and axially expanded when in the accommodated configuration.

In some embodiments, an axial distance separating the anterior lens and the posterior lens can be increased when the accommodating intraocular lens is in the accommodated configuration compared to when the accommodating intraocular lens is in the non-accommodated configuration. For example, the axial distance separating the anterior lens and the posterior lens can be increased by about 500 μm when the accommodating intraocular lens is in the accommodated configuration compared to when the accommodating intraocular lens is in the non-accommodated configuration.

In some embodiments, the mechanical structure comprises a plurality of compressible haptics. Each of the compressible haptics can be substantially shaped as an annulus sector when viewing the accommodating intraocular lens in an anteroposterior direction. The mechanical structure is unfilled with a fluid.

In some embodiments, each of the compressible haptics can comprise an anterior haptic portion, a posterior haptic portion, and a connecting portion configured to couple the anterior haptic portion to the posterior haptic portion. The connecting portion can be fluted or plicated in an anteroposterior direction.

At least one of the anterior lens and the posterior lens can comprise an optical surface profile. For example, the anterior lens can comprise the optical surface profile. Also, for example, the posterior lens can comprise the optical surface profile. In some embodiments, the optical surface profile can be a phase-shifting surface profile or a presbyopia-correcting surface profile.

Also disclosed is an intraocular lens comprising an optic portion and an accommodating mechanical structure coupled to the optic portion. The optic portion can comprise an anterior lens and a posterior lens. The posterior lens can comprise an optical surface profile. In some embodiments, the optical surface profile can be a phase-shifting surface profile or a presbyopia-correcting surface profile.

In addition, disclosed is an intraocular lens, comprising an optic portion and an accommodating mechanical structure coupled to the optic portion. The optic portion can comprise a single optical lens. The single optical lens can comprise an optical surface profile. In some embodiments, the single optical lens can be positioned on an anterior portion of the optic portion. In certain embodiments, the optical surface profile can be a phase-shifting surface profile or a presbyopia-correcting surface profile.

### BRIEF DESCRIPTION OF THE DRAWINGS

FIG. 1A illustrates a top plan view of an embodiment of an accommodating intraocular lens.

FIG. 1B illustrates a front view of the accommodating intraocular lens.

FIG. 1C illustrates a side view of the accommodating intraocular lens.

FIG. 2 illustrates an embodiment of the accommodating intraocular lens implanted within a capsular bag of a subject after a native lens has been removed.

FIG. 3A illustrates the accommodating intraocular lens in a non-accommodated configuration within the capsular bag.

FIG. 3B illustrates the accommodating intraocular lens in an accommodated configuration.

FIG. 4 illustrates a close up view of a phase-shifting lens surface profile of an anterior lens of the accommodating intraocular lens.

### DETAILED DESCRIPTION

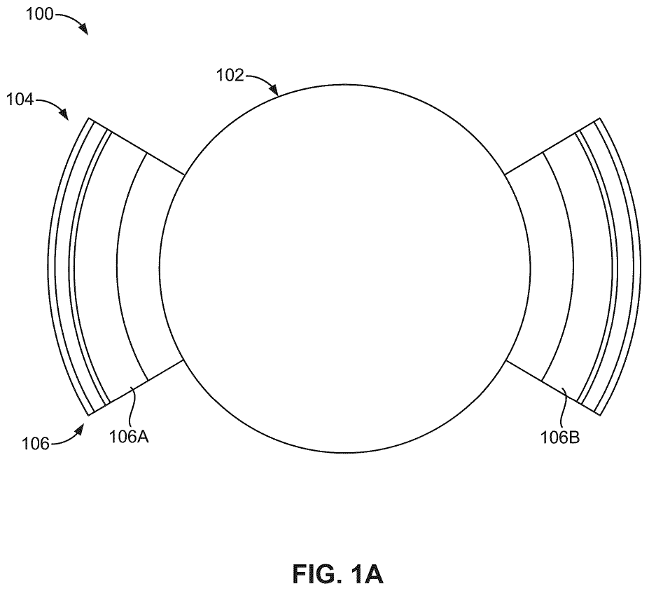

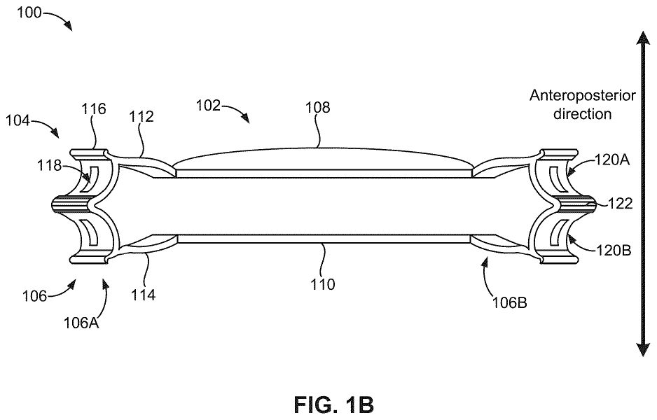

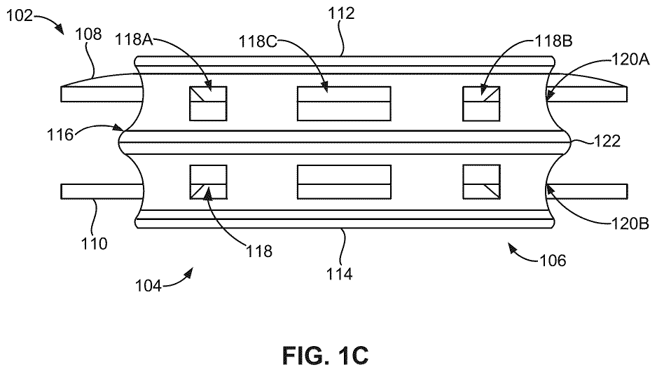

FIG. 1A illustrates a top plan view of an embodiment of an accommodating intraocular lens (AIOL) 100. The AIOL 100 comprises an optic portion 102 and an accommodating mechanical structure 104 coupled to the optic portion 102.

In some embodiments, the accommodating mechanical structure 104 can comprise a plurality of compressible haptics 106. For example, the compressible haptics 106 can comprise a first haptic 106A and a second haptic 106B.

As shown in FIG. 1A, the first haptic 106A can be positioned diametrically opposed to the second haptic 106B. In some embodiments, each of the compressible haptics 106 can be substantially shaped as an annulus sector or partial annulus when viewing the AIOL 100 in a top down (or bottom up) direction or an anteroposterior (or anterior-to-posterior/posterior-to-anterior) direction when the AIOL 100 is implanted within an eye of a subject. In these embodiments, none of the compressible haptics 106 is shaped as a semi-annulus or annulus when viewing the AIOL 100 in an anteroposterior direction (or top down direction when the intraocular lens is not implanted).

In some embodiments, the AIOL 100 can have a total device length as measured from a haptic distal end of the first haptic 106A to the haptic distal end of the second haptic 106B. The total device length can be between about 10.0 mm and about 15.0 mm. For example, the total device length can be between about 11.0 mm and about 13.0 mm.

In some embodiments, the optic portion 102 can have an optic portion diameter. The optic portion diameter can be between about 5.0 mm and 8.0 mm. For example, the optic portion diameter can be about 6.0 mm.

In some embodiments, the AIOL 100 can be made at least in part of a hydrophobic acrylic material. For example, the optic portion 102 can be made at least in part of the hydrophobic acrylic material. In certain embodiments, both the optic portion 102 and the accommodating mechanical structure 104 can be made of the same hydrophobic acrylic material.

As a more specific example, the AIOL 100 can be made at least in part of a hydrophobic acrylate/methacrylate copolymer. In some embodiments, the AIOL 100 can be made at least in part of a hydrophobic acrylic material comprising hydroxyethyl methacrylate (HEMA).

In some embodiments, the AIOL 100 can be made at least in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably, unless otherwise specified.

For example, the cross-linked copolymer can comprise an alkyl acrylate or methacrylate, a fluoro-alkyl acrylate or fluoro-alkyl methacrylate, and a phenyl-alkyl acrylate. In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate.

The final composition of the cross-linked copolymer can also comprise a cross-linker or cross-linking agent, such as ethylene glycol dimethacrylate (EGDMA), a hydroxyl-functional acrylic monomer (e.g., HEMA or hydroxyethyl acrylate (HEA)), certain initiators or initiating agents, and a UV absorber.

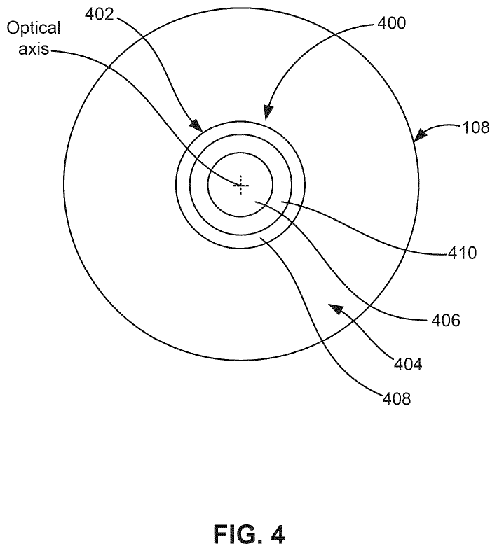

FIG. 1B illustrates a front view of the AIOL 100. As shown in FIG. 1B, the optic portion 102 can comprise an anterior lens 108 and a posterior lens 110. The anterior lens 108 can comprise an optical surface profile. In some embodiments, the optical surface profile can be a phase-shifting surface profile 400 (see, e.g., FIG. 4) or a presbyopia-correcting surface profile.

In alternative embodiments, the posterior lens 110 can comprise the optical surface profile.

In further embodiments, the anterior lens 108 can comprise the optical surface profile and the posterior portion of the optic portion 102 can be a non-optical component or be non-existent.

In some embodiments, the anterior lens 108 is unconnected to the posterior lens 110 other than via the accommodating mechanical structure 104 (including, for example, the first haptic 106A and the second haptic 106B).

As shown in FIG. 1B, the accommodating mechanical structure 104 and the optic portion 102 are unfilled with a fluid. For example, the optic portion 102 does not comprise an optic fluid chamber and none of the haptics 106 comprise a haptic fluid chamber.

The accommodating mechanical structure 104 can comprise a plurality of compressible haptics 106. Each of the compressible haptics 106 can comprise an anterior haptic portion 112, a posterior haptic portion 114, and a connecting portion 116 configured to couple the anterior haptic portion 112 to the posterior haptic portion 114.

In certain embodiments, the anterior haptic portion(s) 112 can be coupled directly to or extend from a periphery of the anterior lens 108 and the posterior haptic portion(s) 114 can be coupled directly to or extend from a periphery of the posterior lens 110. For example, the anterior haptic portion 112 of the first haptic 106A can be coupled directly to or extend from a first position along the periphery of the anterior lens 108 and the anterior haptic portion 112 of the second haptic 106B can be coupled directly to or extend from a second position along the periphery of the anterior lens 108 diametrically opposed to the first position. Also, for example, the posterior haptic portion 114 of the first haptic 106A can be coupled directly to or extend from a first position along the periphery of the posterior lens 110 and the posterior haptic portion 114 of the second haptic 106B can be coupled directly to or extend from a second position along the periphery of the posterior lens 110 diametrically opposed to the first position.

In some embodiments, the connecting portion 116 can comprise cutouts 118 or openings along the connecting portion 116. The cutouts 118 or openings will be discussed in more detail in relation to FIG. 1C.

As shown in FIG. 1B, the connecting portion 116 can be fluted or plicated (i.e., comprising flutes or folds) in an anteroposterior direction. A fluted or plicated surface of the connecting portion 116 can face radially outward from the optic portion 102.

In some embodiments, the connecting portion 116 can comprise a first curved flute portion 120A and a second curved flute portion 120B connected by an elongate fold 122. The first curved flute portion 120A and the second curved flute portion 120B can face radially outward from the optic portion 102.

As will be discussed in more detail in the following sections, at least one of the first curved flute portion 120A and the second curved flute portion 120B can comprise one or more cutouts 118 (e.g., three cutouts 118) or openings. The cutouts 118 or openings can allow the fluted or plicated connecting portion 116 to more easily compress or collapse.

In some embodiments, the anterior lens 108 can be convergent or can be a positive power lens. In these and other embodiments, the posterior lens 110 can be divergent or a negative power lens.

As a more specific example, the anterior lens 108 can have a power of about +30 diopters (D) and the posterior lens 110 can have a power of about −10 D. In this example, a total dioptric power of the optic portion 102 of the AIOL 100 can be about +20 D but this total dioptric power can change when the relative positions of the lenses change as a result of axial movement provided by the accommodating mechanical structure 104.

In some embodiments, the anterior lens 108 of the optic portion 102 can comprise an optical surface profile. For example, the optical surface profile can be a phase-shifting surface profile 400 (see, e.g., FIG. 4) or a presbyopia-correcting surface profile.

In alternative embodiments, the posterior lens 110 can comprise the optical surface profile.

In additional alternative embodiments, the optic portion 102 can comprise only the anterior lens 108 without the posterior lens 110. In further alternative embodiments, the posterior lens 110 can be a non-optical component or a non-optical lens.

FIG. 1C illustrates a close up side view of the AIOL 100. As shown in FIG. 1C, each of the haptics 106 making up the accommodating mechanical structure 104 can comprise an anterior haptic portion 112, a posterior haptic portion 114, and a fluted or plicated connecting portion 116 connecting the anterior haptic portion 112 to the posterior haptic portion 114.

In some embodiments, the anterior haptic portion 112 is unconnected to the posterior haptic portion 114 other than via the connecting portion 116.

In some embodiments, the connecting portion 116 can comprise a first curved flute portion 120A and a second curved flute portion 120B connected by an elongate fold 122. The elongate fold 122 can extend across the entire width of the haptic 106.

The first curved flute portion 120A and the second curved flute portion 120B can face radially outward from the optic portion 102. Although the figures illustrate embodiments of the AIOL 100 comprising two curved flute portions, it is contemplated by this disclosure that the connecting portion 116 can comprise three or more curved flute portions (e.g., three curved flute portions, four curved flute portions, or five or greater curved flute portions) separated by multiple elongate folds 122.

As shown in FIG. 1C, the first curved flute portion 120A and the second curved flute portion 120B can each comprise one or more cutouts 118 (e.g., three cutouts 118) or openings. The cutouts 118 or openings can allow the fluted or plicated connecting portion 116 to more easily compress or collapse.

As a more specific example, each of or at least one of the first curved flute portion 120A and the second curved flute portion 120B can comprise three cutouts 118 including a first lateral cutout 118A, a second lateral cutout 118B, and a middle cutout 118C in between the first lateral cutout 118A and the second lateral cutout 118B. The middle cutout 118C can have a greater width dimension than either the first lateral cutout 118A or the second lateral cutout 118B. In some embodiments, the first lateral cutout 118A, the second lateral cutout 118B, and the middle cutout 118C can all have the same cutout height. In other embodiments, one of the first lateral cutout 118A, the second lateral cutout 118B, or the middle cutout 118C can have a different cutout height than the other cutouts or openings.

Although FIG. 1C illustrates the connecting portion 116 comprising rounded or curved flutes or pleats in an anteroposterior direction, it is contemplated by this disclosure that the connecting portion 116 can comprise zig-zagged, accordion, wave-shaped, or other types of flutes or pleats.

FIG. 1C also illustrates that, in some embodiments, the maximum height or thickness of the haptic 106 (or the maximum height or thickness of the accommodating mechanical structure 104) can be greater than a maximum height of the optic portion 102. In certain embodiments, the maximum height or thickness of the haptic 106 (or the maximum height or thickness of the accommodating mechanical structure 104) can be substantially equivalent to the maximum height of the optic portion 102.

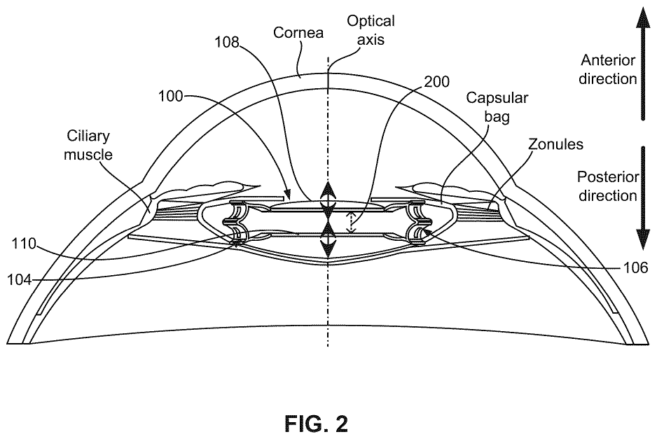

Moreover, FIG. 1C illustrates that a void or open space can separate the anterior lens 108 from the posterior lens 110 except where the anterior lens 108 is connected to the posterior lens 110 via the accommodating mechanical structure 104 (including the haptics 106). For example, a void or open space can separate the anterior lens 108 from the posterior lens 110 along an optical axis of the optic portion 102 (see, also, FIG. 2).

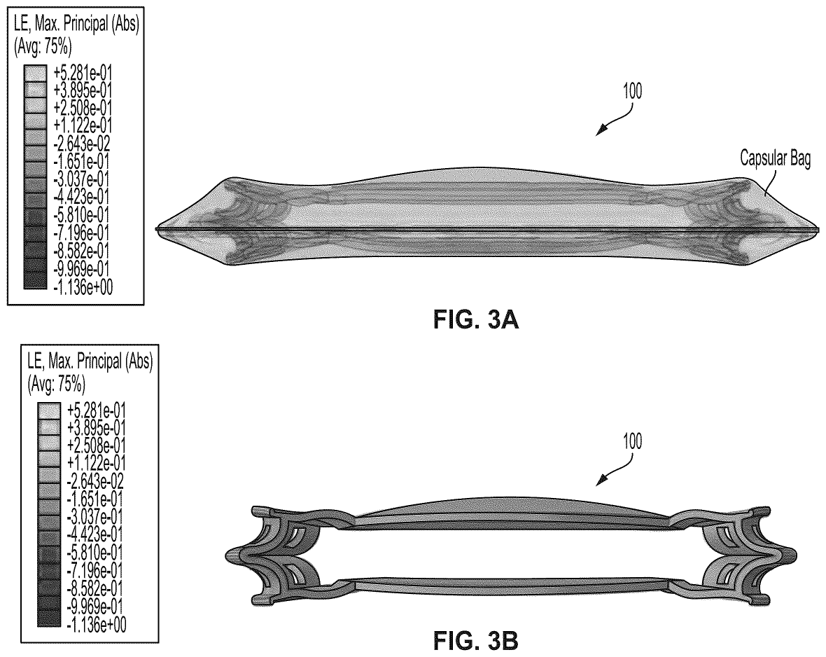

FIG. 2 illustrates an embodiment of the AIOL 100 implanted within a capsular bag of a subject after a native lens has been removed. As shown in FIG. 2, the elastic capsular bag is connected to zonules, which are, in turn, connected to the eye's ciliary muscles. When the ciliary muscles relax, the zonules are stretched and this stretching pulls the capsular bag in a generally radially outward direction due to the generally equatorial connection location between the capsular bag and the zonules. This zonular stretching causes the capsular bag to elongate and become thinner. As a result of the capsular bag becoming more elongate and thinner, the AIOL 100 within the capsular bag becomes axially deformed with the accommodating mechanical structure 104 (including the haptics 106) becoming more axially compressed (see, e.g., FIG. 3A). More specifically, the fluted or plicated connecting portion 116 of each of the haptics 106 can fold down or become more axially compressed.

When in this configuration, the AIOL 100 can be considered to be in an unaccommodated or disaccommodated state. When in this configuration or state, an axial distance 200 separating the anterior lens 108 and the posterior lens 110 can be decreased compared to when the AIOL 100 is in an accommodated configuration or state (see FIG. 3B).

However, when the ciliary muscles contract (such as when the eye is attempting to focus on near objects), the radially inner portion of the ciliary muscles move radially inward, causing radial forces to increase and the zonules to slacken. The slack in the zonules allows the capsular bag to contract and the axial forces applied to the capsular bag to decrease. This causes energy stored in the compressed accommodating mechanical structure 104 (including the compressed haptics 106, more specifically, the compressed fluted or plicated connecting portions 116) to be released and the compressed accommodating mechanical structure 104 (the compressed haptics 106) to axially expand. When in this configuration, the AIOL 100 can be considered to be in an accommodated state. When in this configuration or state, an axial distance 200 separating the anterior lens 108 and the posterior lens 110 can be increased compared to when the AIOL 100 is in an unaccommodated configuration or state (see FIG. 3A).

As previously discussed, since the anterior lens 108 is convergent and the posterior lens 110 is divergent, the anterior movement of the anterior lens 108 toward the cornea increases the total power of the eye. Similarly, posterior movement of the posterior lens 110 toward the retina also increases the total power of the eye. With this mechanism, the total dioptric power of the AIOL 100 can be increased during the accommodative process.

FIG. 2 also illustrates that a method of correcting presbyopia in a subject can comprise implanting the AIOL 100 disclosed herein in an eye of the subject.

The method can comprise removing at least part of an anterior capsular wall of the capsular bag of the subject to form a capsulorhexis. The term “capsulorhexis” can refer to both the circular opening formed along the anterior capsular wall of the capsular bag or the technique of forming such an opening. In some embodiments, the capsulorhexis can be formed using a cystotome needle, capsular forceps, or a combination thereof. In other embodiments, the capsulorhexis can be formed using a femtosecond laser. The capsulorhexis can be formed as part of a phacoemulsification procedure. In other embodiments, the capsulorhexis can be formed as part of a manual small incision cataract surgery (MSICS) procedure.

The method can also comprise removing cellular material from within the capsular bag. This step can be performed after the native crystalline lens of the patient is removed. For example, this step can be performed after the native crystalline lens is emulsified using an ultrasonic phacoemulsification probe and aspirated from the capsular bag.

The method can further comprise introducing the AIOL 100 disclosed herein into the capsular bag through the capsulorhexis. As previously disclosed, the AIOL 100 can comprise the optic portion 102 and the accommodating mechanical structure 104 coupled to the optic portion 102 and configured to be compressible and expandable. The optic portion 102 can comprise the anterior lens 108 and the posterior lens 110. In some embodiments, the anterior lens 108 can be convergent (a positive power or convex lens) and the posterior lens 110 can be divergent (a negative power or concave lens).

FIG. 3A illustrates the AIOL 100 in a non-accommodated configuration or state within an elongated and stretched capsular bag. As shown in FIG. 3A, the AIOL 100 within the stretched capsular bag is axially deformed with the accommodating mechanical structure 104 (the haptics 106) axially compressed. When in this configuration, the axial distance 200 (see FIG. 2) separating the anterior lens 108 and the posterior lens 110 is decreased compared to when the AIOL 100 is in an accommodated configuration or state (see FIG. 3B).

In some embodiments, the axial distance 200 separating the anterior lens 108 and the posterior lens 110 (see FIG. 2) can be decreased by about 500 μm. In other embodiments, the axial distance 200 separating the anterior lens 108 and the posterior lens 110 can be decreased by about 100 μm, 200 μm, 300 μm, or 600 μm or greater.

FIG. 3B illustrates the AIOL 100 in an accommodated configuration. The AIOL 100 can achieve this configuration when the ciliary muscles contract (such as when the eye is attempting to focus on near objects) and the zonules to slacken. The slack in the zonules allows the capsular bag to contract and the axial forces applied to the capsular bag to decrease. The capsular bag is not shown in FIG. 3B for ease of viewing.

When the axial forces applied to the capsular bag decreases, the energy stored in the compressed accommodating mechanical structure 104 (haptics 106) is released and the accommodating mechanical structure 104 (haptics 106) axially expands. When in this configuration or state, the axial distance 200 (see, e.g., FIG. 2) separating the anterior lens 108 and the posterior lens 110 is increased compared to when the AIOL 100 is in an unaccommodated configuration or state (see FIG. 3A).

In some embodiments, the axial distance 200 separating the anterior lens 108 and the posterior lens 110 (see FIG. 2) can be increased by about 500 μm. In other embodiments, the axial distance 200 separating the anterior lens 108 and the posterior lens 110 can be increased by about 100 μm, 200 μm, 300 μm, or 600 μm or greater.

In both FIGS. 3A and 3B, the absolute maximum strains are shown in the legends.

FIG. 4 illustrates a close-up view of a phase-shifting lens surface profile 400 of an anterior lens 108 of the AIOL 100. The phase-shifting lens surface profile 400 can be defined on an external optical surface of the anterior lens 108.

In some embodiments, the phase-shifting lens surface profile 400 can comprise a central area or structure comprising a plurality of zones or steps. In certain embodiments, the phase-shifting lens surface profile 400 can stretch, shape, or split light into one or more extended or stretched focal points and/or into multiple discrete foci. In these embodiments, the AIOL 100 can be considered an accommodating extended depth of focus intraocular lens.

As shown in FIG. 4, in some embodiments, the anterior lens 108 can comprise a first zone 402 extending from the optical axis to a first radial boundary and a second zone 404 extending from the first radial boundary to the edge of the anterior lens 108. Additionally, the first zone 402 can include an inner region 406 and an outer region 408 separated by a phase shift feature 410.

In some embodiments, the phase shift feature 410 can include a ridge projecting anteriorly from the anterior lens 108. The ridge can have an increased thickness relative to an adjacent portion of the inner region 406. The ridge can also comprise a ridge surface, a first phase shift step extending outwardly from the inner region 406 to the ridge surface, and a second phase shift step extending inwardly from the ridge surface to the outer region 408.

In some embodiments, the inner region 406 and the outer region 408 can have the same optical power. Moreover, a step height of the first phase shift step can be greater than a step height of the second phase shift step.

One technical problem faced by the applicant is how to design an IOL that provides vision at various distances (e.g., distance vision all the way to near vision) without the side effects and visual disturbances that typically accompany multifocal diffractive lenses such as halos, reduced contrast sensitivity, and compromises in image quality. One technical solution discovered and developed by the applicant is the accommodating IOL 100 disclosed herein comprising an optic portion 102 having an anterior lens 108 with a non-diffractive wavefront-shaping lens surface profile combined with an accommodating mechanical structure 104 that can axially move the anterior lens 108 with the non-diffractive wavefront-shaping lens surface profile to allow for a full range of vision (i.e., distance vision all the way to near vision). Thus, the benefits of a more traditional diffractive multifocal lens (with discrete near, intermediate, and distance focal points that allow for vision at all distances) can be achieved without the side effects (e.g., halos, reduced contrast sensitivity, and compromises in image quality) that typically accompany these types of traditional multifocal diffractive lenses.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having,” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about,” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An intraocular lens, comprising:
an optic portion comprising an anterior lens and a posterior lens,
wherein the anterior lens comprises an optical surface profile; and
an accommodating mechanical structure coupled to the optic portion.

2. The intraocular lens of claim 1, wherein the anterior lens is unconnected to the posterior lens other than via the accommodating mechanical structure.

3. The intraocular lens of claim 1, wherein the intraocular lens has an accommodated configuration and a non-accommodated configuration, wherein the intraocular lens is axially compressed when in the non-accommodated configuration, and wherein the intraocular lens is axially expanded when in the accommodated configuration.

4. The intraocular lens of claim 3, wherein an axial distance separating the anterior lens and the posterior lens is increased when the intraocular lens is in the accommodated configuration compared to when the intraocular lens is in the non-accommodated configuration.

5. The intraocular lens of claim 4, wherein the axial distance separating the anterior lens and the posterior lens is increased by about 500 μm when the intraocular lens is in the accommodated configuration compared to when the intraocular lens is in the non-accommodated configuration.

6. The intraocular lens of claim 1, wherein the accommodating mechanical structure is unfilled with a fluid.

7. The intraocular lens of claim 1, wherein the accommodating mechanical structure comprises a plurality of compressible haptics.

8. The intraocular lens of claim 7, wherein the compressible haptics comprises a first compressible haptic and a second compressible haptic, and wherein the first compressible haptic is positioned diametrically opposed to the second compressible haptic.

9. The intraocular lens of claim 7, wherein each of the compressible haptics is substantially shaped as an annulus sector when viewing the intraocular lens in an anteroposterior direction.

10. The intraocular lens of claim 7, wherein none of the compressible haptics is shaped as a semi-annulus when viewing the intraocular lens in an anteroposterior direction.

11. The intraocular lens of claim 7, wherein each of the compressible haptics comprises an anterior haptic portion, a posterior haptic portion, and a connecting portion configured to couple the anterior haptic portion to the posterior haptic portion.

12. The intraocular lens of claim 11, wherein the connecting portion comprises cutouts or openings along the connecting portion.

13. The intraocular lens of claim 11, wherein the connecting portion is fluted or plicated in an anteroposterior direction.

14.-20. (canceled)

21. An accommodating intraocular lens, comprising:
an optic portion comprising an anterior lens and a posterior lens; and
a plurality of haptics coupled to the optic portion,
wherein the haptics are configured to be compressible and expandable in an anteroposterior direction.

22.-40. (canceled)

41. An accommodating intraocular lens, comprising:
an optic portion comprising an anterior lens and a posterior lens; and
a plurality of haptics coupled to the optic portion,
wherein at least one of the haptics is substantially shaped as an annulus sector.

42.-60. (canceled)

61. An accommodating intraocular lens, comprising:
an optic portion comprising an anterior lens and a posterior lens; and
a plurality of haptics coupled to the optic portion,
wherein at least one of the haptics comprises an anterior haptic portion and a posterior haptic portion, wherein the anterior haptic portion is coupled to the posterior haptic portion by a connecting portion, and wherein the connecting portion is fluted or plicated in an anteroposterior direction.

62.-80. (canceled)

81. An intraocular lens, comprising:
an optic portion comprising an anterior lens and a posterior lens,
wherein the anterior lens is a convergent lens and the posterior lens is a divergent lens; and
an accommodating mechanical structure coupled to the optic portion.

82.-90. (canceled)

91. A method of correcting presbyopia in a subject, comprising:
implanting an accommodating intraocular lens in the subject, wherein the accommodating intraocular lens comprises:
an optic portion comprising an anterior lens and a posterior lens, and
a mechanical structure coupled to the optic portion and configured to be compressible and expandable.

92.-100. (canceled)

101. An intraocular lens, comprising:
an optic portion comprising an anterior lens and a posterior lens, wherein the posterior lens comprises an optical surface profile; and
an accommodating mechanical structure coupled to the optic portion.

102. (canceled)

103. An intraocular lens, comprising:
an optic portion comprising a single optical lens, wherein the single optical lens comprises an optical surface profile; and
an accommodating mechanical structure coupled to the optic portion.

104.-105. (canceled)
